(19) United States
(12) Patent Application Publication
(10) Pub. No.: US 2018/0169169 A1
(43) Pub. Date: Jun. 21, 2018

(54) HERBAL COMPOSITION FOR TREATMENT OF BACK PAIN

(71) Applicant: Altera International, Ltd., Fort Collins, CO (US)

(72) Inventor: Jay A. ALTMAN, Fort Collins, CO (US)

(21) Appl. No.: 15/576,039

(22) PCT Filed: May 20, 2016
(86) PCT No.: PCT/US16/33495

§ 371 Date: Nov. 21, 2017

Related U.S. Application Data
(60) Provisional application No. 62/164,980, filed on May 21, 2015.

(51) Int. Cl. A61K 36/53; A61K 36/34; A61K 36/28; A61K 36/30; A61K 36/9068

(52) U.S. Cl. CPC A61K 36/53; A61K 36/34; A61K 36/9068; A61K 36/30; A61K 36/28

(57) ABSTRACT

The present invention encompasses compositions and methods for reducing back pain in a subject.

### CROSS REFERENCE TO RELATED APPLICATIONS

This application claims the benefit of U.S. Provisional Application No. 62/164,980, filed May 21, 2015, the disclosure of which is hereby incorporated by reference in its entirety.

### FIELD OF THE INVENTION

The invention encompasses compositions and methods for reducing back pain in a subject.

### BACKGROUND OF THE INVENTION

Back pain results in decreased range of motion and limited flexibility of the thoracolumbar spine, and it leads to performance issues. Ancillary therapeutics and alternative therapies are often administered to patients with back pain. Such treatments often fail to provide sufficient pain management in the affected subject.

Equine back problems are common, particularly in performance horses. The conditions involved can be primary or can result from lameness, ill-fitting tack, or even inadequate schooling. Generally, veterinary practitioners and farriers advocate rest as the most effective remedy for back troubles. For some types of thoracolumbar complaint, this often proves to be all that is needed and no other therapy is necessary. However, in many horses pain remains following the prescribed rest period. Thus, new methods are needed to treat equine with refractory back pain.

### SUMMARY OF THE INVENTION

In an aspect, the disclosure provides a method for reducing back pain in a subject. The method comprises: identifying a subject in need of treatment for back pain, and administering composition A to the subject, wherein composition A comprises witch hazel extract and leaves, arnica (Arnica montana), rosemary (Rosmarinus officinalis), lavender (Lavendula officinalis), and lobelia (Lobelia inflata).

### DETAILED DESCRIPTION OF THE INVENTION

The present invention provides methods of reducing back pain in a subject. In particular embodiments, the present invention provides methods of reducing or eliminating refractory back pain. In additional embodiments, the present invention provides methods of reducing or eliminating back pain that is still present following a rest period. Generally speaking, such methods comprise daily administration of Composition A.

### I. Back Pain

One embodiment of the present invention encompasses a method of reducing back pain. Another embodiment of the present invention encompasses a method of reducing refractory back pain. “Refractory back pain” is defined as back pain that has not resolved despite a period of rest and/or standard treatments or back pain that initially responded to standard treatment but is now resistant to or less responsive to standard treatment as evidenced by the return of back pain. “Not resolved” may mean that the subject experienced relief of back pain but not total resolution of pain symptoms. Non-limiting examples of standard treatments for back pain include corticosteroid injection, NSAID administration (systemic or topical), muscle relaxants, mesotherapy, chiropractics, acupuncture, estrone sulfate (corticosteroid), hyaluronate injection, extracorporeal shock wave therapy (ESWT), heat therapy, magnetic therapy, ceramic therapy and surgery. In certain embodiments, refractory back pain may be back pain that has not resolved following a rest period of about 5 or more days. For example, refractory back pain may be back pain that has not resolved following a rest period of about 5 or more days, about 10 or more days, about 15 or more days, about 20 or more days, about 25 or more days, about 30 or more days, about 35 or more days, or about 40 or more days. In certain embodiments, refractory back pain may be back pain that has not resolved following a rest period of about 5, 6, 7, 8, 9, 10, 11, 12, 13, 14, 15, 16, 17, 18, 19, 20 or 21 days. In other embodiments, refractory back pain may be back pain that has not resolved following a rest period of about 22, 23, 24, 25, 26, 27, 28, 29, 30, 31, 32, 33, 34, 35, 36, 27, 38, 39 or 40 days. In a specific embodiment, refractory back pain may be back pain that has not resolved following a rest period of about 30 days. In certain embodiments, when the subject is an equine, the back pain may be located in the third quadrant of the back of the equine. See the Examples to determine the location of the third quadrant.

Back pain may occur from numerous sources and have various presentations. In certain embodiments, the back pain may be primary back pain. Primary back pain may be due to exercise, overexertion, structural imbalance or, in the case of horses that are ridden, pressure from the weight and balance of the rider. In certain embodiments, primary back pain may be due to sore muscles and soft tissue components that may be strained or sprained. Primary back pain may lead to more severe underlying issues such as arthritis, disk damage such as compression or even disc failure such as degeneration or herniation. In other embodiments, the back pain may be secondary back pain. Secondary back pain may be pain that is caused by an underlying lameness condition. Secondary back pain can either take the form of muscle/soft tissue strain or sprain, or if long standing can even lead to degenerative conditions as described above.

Advantageously, a method of the invention may be used to reduce back pain. Specifically, a method of the invention may be used to reduce refractory back pain. “Reducing” back pain refers to reducing the severity of back pain. In certain embodiments, reducing refractory back pain includes eliminating the back pain. “Eliminating” back pain refers to reducing the severity of back pain to zero. Additionally, a method of the invention may be used to alleviate the pain associated with arthritis and/or disc related problems. A reasonably skilled artisan would or could determine the severity of back pain—for instance, taking a health history or applying pressure to the back. A health history and assessment generally entails obtaining a complete performance history and assessing each subject's back through detailed palpitation and visual inspection at rest and in motion. Imaging techniques (i.e. radiographic examination, nuclear imaging, ultrasonographic examination, thermographic examination) may also be used.

Generally speaking, the method comprises the administration of Composition A, as detailed below. In one embodiment, a method of the invention encompasses daily administration of Composition A. Composition A should be administered to the subject suffering from back pain for at least a week, more typically two weeks, and even more typically greater than two weeks. In one embodiment, composition A is administered for at least 1, 2, 3, 4, 5, 6, 7, 8, 9, 10, 11, 12, 13, 14, 15, 16, 17, 18, 19, 20, or more than 20 weeks. In another embodiment, Composition A is administered to the subject daily for the duration of a high risk back pain situation, such as heavy exercising, high stress activities, traveling, training, and competition. In yet another embodiment, Composition A is administered year round. In certain embodiments, Composition A may be administered after exercise. In other embodiments, Composition A may be administered before exercise. In still other embodiments, Composition A may be administered during exercise.

Generally, the method comprises administering, topically, about 0.02 cc of Composition A per square cm. For example, about 0.01, about 0.02, about 0.03, about 0.04, about 0.05, about 0.06, about 0.07, about 0.08, about 0.09, about 0.1, about 0.11, about 0.12, about 0.13, about 0.14, about 0.15, about 0.16, about 0.17, about 0.18, about 0.19, about 0.2 cc of Composition A per square cm may be topically administered. Alternatively, about 0.2, about 0.3, about 0.4, about 0.5, about 0.6, about 0.7, about 0.8, about 0.9 or about 1.0 cc of Composition A per square cm may be topically administered.

As used herein, the term “topical administration” or “topical application” refers to application or administration of a composition of the invention to the skin, for example, by rubbing a cream, ointment, gel, balm, salve, lotion, liniment, poultice, gelotion, shampoo, spray, or the like, directly or indirectly on the surface of the skin to relieve pain. Topical application may also include administration by means of bandages or patches, and the like, in which a composition of the invention is impregnated in or on such devices including transdermal patch devices for transfer to and through the skin.

A method of the invention also comprises identifying a subject in need of treatment for back pain. In one embodiment, “identifying” refers to what a reasonably skilled artisan would or could do to identify a subject suffering from back pain—for instance, taking a health history of the subject as described above. Specifically, when the subject is an equine, the following non-limiting examples may be signs of back pain: objecting to being saddled, being slow to warm up, becoming difficult to shoe, developing a bad attitude, resisting work, displaying abnormal tail swishing, and/or initiating uncharacteristic behavior (such as bolting or running away).

A method of the invention may further comprise administration of standard treatments for back pain in combination with Composition A. Non-limiting examples of standard treatments for back pain include a period of rest, corticosteroid injection, NSAID administration (systemic or topical), muscle relaxants, mesotherapy, chiropractics, acupuncture, estrone sulfate (corticosteroid), hyaluronate injection, extracorporeal shock wave therapy (ESWT), heat therapy, magnetic therapy, ceramic therapy and surgery.

Suitable subjects include, but are not limited to, a human, a livestock animal, a companion animal, a lab animal, and a zoological animal. In one embodiment, the subject may be a rodent, e.g. a mouse, a rat, a guinea pig, etc. In another embodiment, the subject may be a livestock animal. Non-limiting examples of suitable livestock animals may include pigs, cows, horses, goats, sheep, llamas and alpacas. In yet another embodiment, the subject may be a companion animal. Non-limiting examples of companion animals may include pets such as dogs, cats, rabbits, and birds. In yet another embodiment, the subject may be a zoological animal. As used herein, a “zoological animal” refers to an animal that may be found in a zoo. Such animals may include non-human primates, large cats, wolves, and bears. In certain embodiments, the animal is a laboratory animal. Non-limiting examples of a laboratory animal may include rodents, canines, felines, and non-human primates. In other embodiments, the animal is a rodent. Non-limiting examples of rodents may include mice, rats, guinea pigs, etc. In an embodiment, the subject is human. In a specific embodiment, the subject is an equine.

### (a) Composition A

The methods of the invention comprise administration of Composition A. Composition A may be formulated for topical administration. For example, Composition A may be formulated as a cream, ointment, gel, balm, salve, lotion, liniment, poultice, gelotion, shampoo, spray, on to bandages or patches, or any other suitable form. Generally speaking, Composition A may be administered topically.

Composition A is comprised of the following active ingredients: witch hazel extract and leaves, arnica (Arnica montana), rosemary (Rosmarinus officinalis), lavender (Lavendula officinalis), and lobelia (Lobelia inflata). In certain embodiments, comfrey (Symphytum officnate) may be substituted for the lobelia. In other embodiments, ginger (Zingiber officinale) may be substituted for the lavender.

Composition A may further comprise excipients including thickening agents such as xanthum gum; detergents and soaps; conditioners such as tamanu seed oil (Colophyllum tacamchaca), petrolatum (naturally derived), sunflower seed oil (Helianthus annus), aloe vera gel (Aloe barbadensis leaf); packing agents such as Bentonite (bowie type clay); skin softeners and conditioners such as Almond Oil, Shea butter, Bees Wax (human based preparations); and emulsifiers and preservatives.

The additional excipients may depend upon the desired formulation. For example, thickening agents may be used in the gelotion formulation, detergents and soaps may be used in the shampoo formulation, conditioners may be used in the salve formulation, and packing agents may be used in the poultice formulation.

In a specific embodiment, Composition A comprises SORE NO-MORE®, which is commercially available through Arenus®.

### Definitions

As used herein, “equine” refers to a member of the genus Equus, including E. africanus, E. ferus, E. ferus caballus, E. grevyi, E. hemionus, E. kiang, E. quagga, and E. zebra. In an exemplary embodiment, “equine” refers to E. ferus caballus.

### EXAMPLES

The following examples are included to demonstrate preferred embodiments of the invention. It should be appreciated by those of skill in the art that the techniques disclosed in the examples that follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1. Methods of Treating Back Pain in an Equine

Eighteen mature horses were used in a completely randomized 44 d trial to develop a procedure for objectively assessing back pain in horses while also investigating the efficacy of an herbal liniment solution to mitigate muscular pain. Horses began the study after a 30 d rest and were maintained in regular, light work for the entire 44 d study. Horses were randomly assigned to treatment groups which consisted of a commercially available herbal liniment gel solution (SORE NO-MORE® Ultra, Arenus) or a control gel solution (identical gel solution minus active ingredients) applied daily following exercise at 0.02 cc per square cm. Evaluation of back pain was standardized by dividing each horse's back into 4 equal quadrants lengthwise beginning with quadrant 1 at the highest point of the withers and ending with quadrant 4 at the sacroiliac joint. Quadrants extended 17.78 cm ventrally from the spine. The third quadrant (on left and right side) was selected as the area of interest for this study and was clipped in order to maintain consistency over time. Nine data points in this quadrant were selected (top-left, mid-left, bottom-left, top-mid, center, bottom-mid, top-right, mid-right, bottom-right) for weekly evaluation using a force gage pressure meter (operated by same individual throughout trial to maintain consistent technique). A negative pain response was classified as no reaction to the application of 5.9 kg pressure based on previous observations. This data was inverted to create a 0 to 13 pain score based on the amount of pressure applied. Data were analyzed using the PROC MIXED procedure of SAS with main effects tested being treatment, time, and treatmentxtime interaction. The interaction of treatment and time was significant. Treated horses decreased pain score from beginning to end of trial (2.87 decrease; p<0.001) compared to control horses (0.72 decrease; p≤0.18). In summary, use of a force gage pressure meter in conjunction with a standardized evaluation procedure allowed for objective assessment of back pain in horses over time. Additionally, the herbal liniment gel solution was able to mitigate pain response when applied daily over a 44 d period.

TABLE 1
Alogometry Data

| | | | Pounds of pressure applied prior to horse reacting | | | | | | | | | | | | | | | | | |
|---|---|---|---|---|---|---|---|---|---|---|---|---|---|---|---|---|---|---|---|---|
| | | | (13 was the greatest amount of pressure applied and considered no response) | | | | | | | | | | | | | | | | | |
| Horse | Treatment | Day | L1 | L2 | L3 | L4 | L5 | L6 | L7 | L8 | L9 | R1 | R2 | R3 | R4 | R5 | R6 | R7 | R8 | R9 |
| AJ | CON | 2 | 13 | 13 | 13 | 13 | 11 | 13 | 13 | 13 | 10 | 13 | 10 | 13 | 13 | 13 | 13 | 13 | 13 | 13 |
| AJ | CON | 9 | 13 | 13 | 13 | 13 | 11 | 13 | 13 | 13 | 13 | 13 | 13 | 13 | 13 | 13 | 13 | 13 | 13 | 13 |
| AJ | CON | 16 | 13 | 13 | 13 | 13 | 12 | 13 | 13 | 13 | 13 | 13 | 9 | 13 | 13 | 13 | 13 | 13 | 13 | 13 |
| AJ | CON | 23 | 13 | 10 | 10 | 13 | 13 | 13 | 13 | 13 | 13 | 13 | 13 | 13 | 13 | 13 | 13 | 13 | 13 | 13 |
| AJ | CON | 30 | 13 | 13 | 13 | 13 | 13 | 13 | 13 | 13 | 13 | 13 | 13 | 13 | 13 | 13 | 13 | 13 | 13 | 13 |
| AJ | CON | 37 | 13 | 8 | 8 | 10 | 10 | 10 | 13 | 13 | 13 | 10 | 10 | 10 | 10 | 10 | 10 | 10 | 10 | 10 |
| AJ | CON | 44 | 10 | 13 | 13 | 13 | 10 | 13 | 13 | 13 | 13 | 13 | 10 | 13 | 13 | 13 | 13 | 13 | 13 | 13 |
| Ares | SNM | 2 | 13 | 13 | 12 | 13 | 11 | 13 | 10 | 8 | 13 | 13 | 13 | 13 | 9 | 13 | 13 | 12 | 12 | 10 |
| Ares | SNM | 9 | 13 | 13 | 13 | 13 | 13 | 10 | 13 | 12 | 13 | 13 | 13 | 10 | 11 | 13 | 11 | 13 | 13 | 13 |
| Ares | SNM | 16 | 13 | 13 | 6 | 13 | 12 | 8 | 13 | 10 | 8 | 13 | 13 | 10 | 13 | 13 | 6 | 13 | 13 | 13 |
| Ares | SNM | 23 | 13 | 13 | 13 | 13 | 13 | 13 | 13 | 13 | 11 | 13 | 13 | 13 | 13 | 13 | 11 | 13 | 13 | 13 |
| Ares | SNM | 30 | 13 | 13 | 13 | 13 | 13 | 13 | 13 | 13 | 13 | 13 | 13 | 13 | 13 | 13 | 13 | 13 | 13 | 13 |
| Ares | SNM | 37 | 13 | 13 | 13 | 13 | 13 | 11 | 13 | 13 | 13 | 13 | 13 | 13 | 13 | 13 | 7 | 13 | 13 | 13 |
| Ares | SNM | 44 | 13 | 13 | 13 | 13 | 13 | 8 | 13 | 13 | 11 | 13 | 13 | 13 | 13 | 13 | 13 | 13 | 13 | 13 |
| Atticus | SNM | 2 | 6 | 5 | 13 | 8 | 4 | 4 | 6 | 5 | 4 | 10 | 7 | 13 | 7 | 4 | 6 | 6 | 6 | 6 |
| Atticus | SNM | 9 | 13 | 12 | 10 | 10 | 7 | 9 | 13 | 8 | 13 | 13 | 8 | 13 | 8 | 10 | 8 | 13 | 13 | 13 |
| Atticus | SNM | 16 | 12 | 12 | 13 | 12 | 12 | 12 | 11 | 13 | 10 | 12 | 12 | 13 | 12 | 13 | 12 | 12 | 13 | 13 |
| Atticus | SNM | 23 | 13 | 13 | 13 | 10 | 11 | 11 | 10 | 9 | 11 | 12 | 12 | 13 | 12 | 13 | 12 | 13 | 13 | 13 |
| Atticus | SNM | 30 | 13 | 11 | 13 | 13 | 13 | 11 | 13 | 11 | 13 | 13 | 12 | 13 | 13 | 13 | 12 | 13 | 13 | 13 |
| Atticus | SNM | 37 | 9 | 13 | 13 | 10 | 13 | 13 | 13 | 13 | 13 | 9 | 10 | 10 | 10 | 13 | 13 | 13 | 10 | 13 |
| Atticus | SNM | 44 | 13 | 10 | 10 | 9 | 9 | 9 | 13 | 10 | 10 | 8 | 8 | 8 | 8 | 6 | 10 | 13 | 10 | 13 |
| Champ | CON | 2 | 4 | 6 | 9 | 3 | 3 | 5 | 4 | 5 | 5 | 8 | 5 | 8 | 4 | 5 | 8 | 5 | 4 | 10 |
| Champ | CON | 9 | 7 | 7 | 10 | 8 | 7 | 8 | 11 | 9 | 9 | 8 | 13 | 8 | 8 | 6 | 12 | 11 | 13 | 13 |
| Champ | CON | 16 | 9 | 9 | 12 | 10 | 13 | 13 | 13 | 13 | 13 | 7 | 7 | 11 | 9 | 12 | 12 | 13 | 13 | 12 |
| Champ | CON | 23 | 10 | 10 | 10 | 7 | 13 | 7 | 13 | 12 | 9 | 8 | 8 | 10 | 6 | 13 | 13 | 12 | 10 | 13 |
| Champ | CON | 30 | 9 | 8 | 8 | 8 | 6 | 6 | 8 | 13 | 10 | 8 | 8 | 8 | 8 | 6 | 6 | 8 | 13 | 13 |
| Champ | CON | 37 | 8 | 8 | 8 | 10 | 10 | 10 | 10 | 13 | 10 | 10 | 10 | 10 | 10 | 10 | 10 | 10 | 10 | 13 |
| Champ | CON | 44 | 10 | 11 | 13 | 10 | 10 | 10 | 9 | 11 | 11 | 10 | 10 | 13 | 8 | 13 | 10 | 13 | 10 | 13 |
| Dublin | SNM | 2 | 9 | 5 | 7 | 5 | 4 | 5 | 4 | 5 | 7 | 13 | 8 | 7 | 7 | 7 | 5 | 5 | 6 | 6 |
| Dublin | SNM | 9 | 8 | 9 | 7 | 10 | 10 | 12 | 7 | 9 | 7 | 10 | 9 | 13 | 7 | 10 | 13 | 8 | 7 | 8 |
| Dublin | SNM | 16 | 13 | 9 | 13 | 13 | 7 | 13 | 13 | 8 | 8 | 9 | 8 | 13 | 7 | 8 | 6 | 7 | 7 | 13 |
| Dublin | SNM | 23 | 12 | 12 | 12 | 12 | 10 | 12 | 11 | 13 | 13 | 13 | 10 | 13 | 10 | 10 | 10 | 13 | 10 | 13 |
| Dublin | SNM | 30 | 13 | 13 | 13 | 8 | 13 | 13 | 10 | 10 | 10 | 8 | 8 | 8 | 8 | 8 | 8 | 8 | 8 | 6 |
| Dublin | SNM | 37 | 3 | 13 | 5 | 9 | 13 | 13 | 10 | 7 | 7 | 3 | 4 | 7 | 7 | 5 | 5 | 5 | 7 | 5 |
| Dublin | SNM | 44 | 11 | 10 | 11 | 11 | 11 | 10 | 13 | 13 | 13 | 11 | 10 | 11 | 10 | 11 | 11 | 13 | 13 | 11 |
| Fatima | CON | 2 | 11 | 7 | 8 | 9 | 13 | 11 | 13 | 7 | 13 | 13 | 13 | 13 | 13 | 11 | 13 | 13 | 10 | 13 |
| Fatima | CON | 9 | 12 | 13 | 13 | 10 | 13 | 13 | 13 | 13 | 12 | 9 | 13 | 9 | 13 | 13 | 13 | 13 | 13 | 12 |
| Fatima | CON | 16 | 9 | 12 | 7 | 12 | 13 | 13 | 13 | 12 | 13 | 12 | 9 | 11 | 13 | 13 | 13 | 13 | 13 | 13 |
| Fatima | CON | 23 | 12 | 13 | 11 | 12 | 13 | 13 | 13 | 13 | 11 | 13 | 13 | 10 | 13 | 11 | 12 | 13 | 12 | 13 |
| Fatima | CON | 30 | 12 | 9 | 10 | 11 | 12 | 13 | 10 | 13 | 10 | 10 | 10 | 8 | 10 | 10 | 13 | 13 | 10 | 9 |
| Fatima | CON | 37 | 8 | 10 | 10 | 13 | 10 | 10 | 13 | 13 | 13 | 7 | 6 | 7 | 13 | 11 | 13 | 13 | 8 | 13 |
| Fatima | CON | 44 | 10 | 13 | 10 | 13 | 10 | 13 | 13 | 13 | 13 | 10 | 13 | 13 | 13 | 10 | 13 | 13 | 13 | 13 |
| Flick | CON | 2 | 9 | 13 | 11 | 11 | 11 | 13 | 10 | 8 | 8 | 13 | 10 | 13 | 10 | 10 | 13 | 13 | 13 | 13 |
| Flick | CON | 9 | 11 | 13 | 11 | 9 | 7 | 8 | 10 | 7 | 7 | 13 | 10 | 13 | 13 | 11 | 13 | 10 | 9 | 6 |
| Flick | CON | 16 | 13 | 13 | 13 | 12 | 13 | 10 | 13 | 13 | 10 | 12 | 13 | 13 | 10 | 10 | 13 | 13 | 13 | 13 |
| Flick | CON | 23 | 13 | 13 | 13 | 11 | 11 | 12 | 13 | 11 | 12 | 11 | 13 | 13 | 9 | 12 | 11 | 13 | 8 | 12 |
| Flick | CON | 30 | 13 | 13 | 13 | 13 | 10 | 13 | 13 | 13 | 13 | 13 | 10 | 10 | 13 | 10 | 13 | 13 | 13 | 10 |
| Flick | CON | 37 | 10 | 13 | 13 | 10 | 10 | 10 | 10 | 9 | 7 | 9 | 8 | 8 | 7 | 10 | 11 | 10 | 10 | 10 |
| Flick | CON | 44 | 13 | 10 | 10 | 13 | 10 | 13 | 13 | 8 | 11 | 13 | 13 | 10 | 10 | 10 | 13 | 13 | 10 | 10 |
| Fury | CON | 2 | 10 | 13 | 11 | 10 | 6 | 8 | 13 | 13 | 13 | 13 | 10 | 13 | 11 | 8 | 13 | 13 | 11 | 13 |
| Fury | CON | 9 | 13 | 13 | 11 | 7 | 5 | 9 | 13 | 13 | 13 | 10 | 9 | 10 | 11 | 12 | 9 | 13 | 11 | 7 |
| Fury | CON | 16 | 13 | 12 | 13 | 12 | 12 | 13 | 13 | 13 | 13 | 13 | 12 | 13 | 13 | 13 | 10 | 13 | 12 | 10 |
| Fury | CON | 23 | 13 | 13 | 13 | 13 | 13 | 13 | 13 | 13 | 13 | 13 | 13 | 11 | 11 | 13 | 12 | 13 | 12 | 13 |
| Fury | CON | 30 | 13 | 13 | 13 | 13 | 8 | 10 | 13 | 13 | 13 | 13 | 13 | 13 | 13 | 13 | 13 | 13 | 13 | 13 |
| Fury | CON | 37 | 9 | 13 | 11 | 11 | 13 | 13 | 13 | 13 | 13 | 13 | 13 | 13 | 13 | 13 | 13 | 13 | 13 | 13 |
| Fury | CON | 44 | 13 | 13 | 13 | 10 | 10 | 10 | 13 | 13 | 13 | 13 | 13 | 13 | 10 | 13 | 10 | 13 | 13 | 13 |
| Glory | SNM | 2 | 8 | 3 | 7 | 5 | 3 | 5 | 3 | 3 | 6 | 6 | 6 | 13 | 4 | 3 | 5 | 3 | 1 | 6 |
| Glory | SNM | 9 | 11 | 6 | 8 | 7 | 5 | 5 | 13 | 8 | 8 | 9 | 7 | 11 | 13 | 10 | 8 | 13 | 8 | 7 |
| Glory | SNM | 16 | 8 | 8 | 8 | 8 | 8 | 8 | 13 | 8 | 8 | 8 | 8 | 8 | 13 | 6 | 7 | 13 | 13 | 13 |
| Glory | SNM | 23 | 10 | 10 | 10 | 11 | 8 | 4 | 10 | 8 | 8 | 8 | 8 | 8 | 8 | 10 | 6 | 13 | 8 | 8 |
| Glory | SNM | 30 | 13 | 13 | 13 | 13 | 13 | 13 | 13 | 13 | 13 | 13 | 13 | 13 | 13 | 13 | 13 | 13 | 10 | 13 |
| Glory | SNM | 37 | 8 | 10 | 10 | 13 | 10 | 10 | 13 | 13 | 13 | 10 | 13 | 13 | 13 | 13 | 13 | 13 | 13 | 13 |
| Glory | SNM | 44 | 11 | 9 | 13 | 13 | 13 | 13 | 13 | 13 | 11 | 13 | 13 | 13 | 13 | 13 | 13 | 13 | 13 | 13 |
| Goldie | SNM | 2 | 13 | 13 | 13 | 11 | 8 | 8 | 13 | 13 | 13 | 13 | 13 | 13 | 11 | 11 | 13 | 9 | 12 | 13 |
| Goldie | SNM | 9 | 11 | 12 | 13 | 13 | 9 | 9 | 13 | 13 | 10 | 12 | 11 | 10 | 11 | 7 | 9 | 11 | 8 | 8 |
| Goldie | SNM | 16 | 13 | 12 | 13 | 11 | 11 | 13 | 13 | 13 | 13 | 13 | 13 | 13 | 10 | 8 | 13 | 13 | 13 | 13 |
| Goldie | SNM | 23 | 13 | 13 | 13 | 13 | 10 | 13 | 13 | 13 | 13 | 13 | 13 | 13 | 13 | 13 | 13 | 13 | 13 | 13 |
| Goldie | SNM | 30 | 13 | 13 | 13 | 13 | 13 | 13 | 13 | 13 | 13 | 13 | 10 | 13 | 13 | 13 | 13 | 13 | 13 | 13 |
| Goldie | SNM | 37 | 13 | 13 | 13 | 13 | 10 | 13 | 13 | 10 | 13 | 13 | 10 | 10 | 13 | 10 | 13 | 13 | 13 | 13 |
| Goldie | SNM | 44 | 13 | 13 | 13 | 10 | 10 | 13 | 13 | 13 | 13 | 13 | 13 | 13 | 13 | 13 | 13 | 13 | 13 | 13 |
| Gypsy | CON | 2 | 12 | 9 | 13 | 9 | 9 | 9 | 9 | 8 | 10 | 13 | 8 | 12 | 8 | 8 | 8 | 8 | 10 | 10 |
| Gypsy | CON | 9 | 9 | 8 | 10 | 5 | 9 | 13 | 12 | 13 | 13 | 13 | 8 | 10 | 13 | 11 | 13 | 13 | 13 | 13 |
| Gypsy | CON | 16 | 5 | 5 | 9 | 13 | 6 | 12 | 13 | 13 | 13 | 5 | 3 | 13 | 10 | 5 | 13 | 13 | 13 | 12 |
| Gypsy | CON | 23 | 10 | 12 | 10 | 13 | 13 | 12 | 13 | 13 | 12 | 8 | 9 | 13 | 13 | 11 | 11 | 13 | 13 | 13 |
| Gypsy | CON | 30 | 5 | 7 | 9 | 13 | 10 | 10 | 13 | 9 | 10 | 13 | 10 | 13 | 13 | 10 | 13 | 13 | 13 | 13 |
| Gypsy | CON | 37 | 8 | 8 | 8 | 8 | 8 | 10 | 13 | 13 | 13 | 8 | 10 | 10 | 10 | 13 | 13 | 13 | 13 | 10 |
| Gypsy | CON | 44 | 9 | 8 | 10 | 10 | 9 | 8 | 9 | 9 | 13 | 13 | 9 | 9 | 10 | 9 | 8 | 11 | 11 | 11 |
| King | SNM | 2 | 13 | 10 | 10 | 13 | 10 | 11 | 10 | 10 | 12 | 13 | 12 | 12 | 13 | 12 | 11 | 13 | 13 | 13 |
| King | SNM | 9 | 13 | 11 | 12 | 13 | 8 | 13 | 10 | 11 | 13 | 10 | 11 | 13 | 11 | 13 | 11 | 13 | 9 | 13 |
| King | SNM | 16 | 13 | 13 | 13 | 13 | 13 | 13 | 13 | 13 | 13 | 10 | 13 | 13 | 10 | 11 | 13 | 10 | 13 | 13 |
| King | SNM | 23 | 13 | 10 | 13 | 13 | 13 | 11 | 13 | 13 | 13 | 11 | 13 | 13 | 13 | 13 | 13 | 13 | 13 | 13 |
| King | SNM | 30 | 13 | 13 | 10 | 12 | 13 | 13 | 13 | 13 | 13 | 10 | 10 | 13 | 13 | 13 | 13 | 13 | 13 | 13 |
| King | SNM | 37 | 9 | 10 | 13 | 10 | 10 | 13 | 13 | 13 | 13 | 10 | 10 | 13 | 9 | 13 | 13 | 8 | 13 | 13 |
| King | SNM | 44 | 13 | 10 | 13 | 10 | 13 | 13 | 13 | 13 | 13 | 10 | 13 | 13 | 13 | 10 | 13 | 13 | 13 | 13 |
| Nova | CON | 2 | 12 | 10 | 13 | 11 | 13 | 13 | 13 | 13 | 13 | 11 | 13 | 13 | 13 | 13 | 13 | 13 | 13 | 10 |
| Nova | CON | 9 | 11 | 8 | 11 | 13 | 13 | 11 | 13 | 13 | 13 | 11 | 10 | 7 | 13 | 6 | 13 | 13 | 11 | 11 |
| Nova | CON | 16 | 13 | 13 | 13 | 11 | 8 | 8 | 13 | 13 | 13 | 8 | 10 | 10 | 13 | 13 | 10 | 13 | 13 | 10 |
| Nova | CON | 23 | 11 | 11 | 11 | 13 | 13 | 13 | 13 | 13 | 10 | 9 | 13 | 10 | 13 | 13 | 13 | 13 | 13 | 13 |
| Nova | CON | 30 | 7 | 7 | 7 | 12 | 7 | 13 | 7 | 10 | 13 | 10 | 8 | 10 | 8 | 10 | 10 | 13 | 13 | 12 |
| Nova | CON | 37 | 10 | 10 | 10 | 10 | 10 | 10 | 10 | 10 | 10 | 9 | 9 | 9 | 13 | 10 | 13 | 13 | 10 | 13 |
| Nova | CON | 44 | 9 | 8 | 13 | 9 | 7 | 10 | 10 | 9 | 13 | 7 | 8 | 7 | 9 | 8 | 9 | 13 | 8 | 10 |
| Pierre | SNM | 2 | 13 | 7 | 7 | 4 | 5 | 4 | 5 | 5 | 6 | 7 | 4 | 6 | 6 | 7 | 6 | 7 | 5 | 5 |
| Pierre | SNM | 9 | 13 | 6 | 8 | 10 | 13 | 10 | 13 | 13 | 10 | 9 | 8 | 9 | 13 | 6 | 7 | 13 | 8 | 7 |
| Pierre | SNM | 16 | 13 | 12 | 7 | 13 | 13 | 8 | 13 | 13 | 8 | 9 | 13 | 8 | 11 | 8 | 10 | 10 | 13 | 8 |
| Pierre | SNM | 23 | 13 | 10 | 11 | 10 | 11 | 12 | 13 | 13 | 10 | 11 | 10 | 9 | 13 | 11 | 10 | 13 | 13 | 13 |
| Pierre | SNM | 30 | 13 | 11 | 11 | 13 | 13 | 9 | 13 | 13 | 13 | 13 | 10 | 10 | 13 | 13 | 13 | 13 | 13 | 9 |
| Pierre | SNM | 37 | 11 | 6 | 10 | 11 | 11 | 13 | 11 | 13 | 13 | 9 | 8 | 8 | 13 | 7 | 10 | 13 | 13 | 10 |
| Pierre | SNM | 44 | 10 | 8 | 13 | 10 | 11 | 10 | 10 | 10 | 10 | 10 | 10 | 11 | 10 | 13 | 10 | 13 | 13 | 11 |
| Quick | CON | 2 | 13 | 13 | 13 | 13 | 13 | 13 | 13 | 13 | 13 | 13 | 13 | 13 | 13 | 13 | 13 | 12 | 12 | 13 |
| Quick | CON | 9 | 13 | 13 | 13 | 13 | 13 | 13 | 13 | 13 | 13 | 13 | 13 | 13 | 13 | 13 | 13 | 13 | 13 | 13 |
| Quick | CON | 16 | 13 | 13 | 13 | 13 | 13 | 13 | 13 | 13 | 13 | 13 | 13 | 13 | 13 | 13 | 13 | 13 | 13 | 13 |
| Quick | CON | 23 | 13 | 13 | 13 | 13 | 13 | 13 | 13 | 13 | 13 | 13 | 13 | 13 | 13 | 10 | 13 | 13 | 13 | 13 |
| Quick | CON | 30 | 13 | 13 | 13 | 13 | 13 | 13 | 13 | 13 | 13 | 13 | 13 | 13 | 13 | 10 | 13 | 13 | 13 | 13 |
| Quick | CON | 37 | 13 | 13 | 13 | 13 | 13 | 13 | 13 | 13 | 13 | 13 | 13 | 13 | 13 | 13 | 13 | 13 | 13 | 13 |
| Quick | CON | 44 | 13 | 11 | 13 | 13 | 13 | 13 | 13 | 13 | 13 | 10 | 13 | 13 | 13 | 13 | 13 | 10 | 13 | 13 |
| Red | SNM | 2 | 13 | 13 | 13 | 12 | 10 | 10 | 13 | 13 | 13 | 13 | 13 | 10 | 11 | 11 | 11 | 9 | 10 | 9 |
| Red | SNM | 9 | 13 | 10 | 8 | 7 | 7 | 13 | 13 | 7 | 8 | 8 | 13 | 13 | 7 | 13 | 8 | 9 | 10 | 12 |
| Red | SNM | 16 | 13 | 10 | 10 | 10 | 7 | 9 | 13 | 12 | 10 | 12 | 13 | 10 | 10 | 8 | 13 | 13 | 13 | 13 |
| Red | SNM | 23 | 13 | 13 | 13 | 13 | 13 | 13 | 13 | 13 | 13 | 13 | 10 | 13 | 13 | 10 | 13 | 13 | 13 | 13 |
| Red | SNM | 30 | 13 | 13 | 13 | 13 | 13 | 13 | 13 | 13 | 13 | 13 | 10 | 13 | 13 | 13 | 13 | 13 | 13 | 13 |
| Red | SNM | 37 | 13 | 13 | 13 | 10 | 13 | 13 | 13 | 13 | 13 | 8 | 13 | 13 | 13 | 10 | 13 | 13 | 13 | 13 |
| Red | SNM | 44 | 13 | 13 | 13 | 13 | 13 | 13 | 13 | 13 | 13 | 13 | 13 | 13 | 13 | 13 | 13 | 13 | 13 | 13 |
| Sneaks | CON | 2 | 9 | 13 | 13 | 8 | 6 | 13 | 10 | 10 | 13 | 12 | 9 | 10 | 5 | 5 | 5 | 8 | 7 | 5 |
| Sneaks | CON | 9 | 10 | 13 | 13 | 10 | 8 | 13 | 10 | 10 | 13 | 13 | 10 | 10 | 6 | 6 | 7 | 10 | 10 | 5 |
| Sneaks | CON | 16 | 13 | 13 | 13 | 13 | 10 | 10 | 13 | 13 | 13 | 13 | 13 | 9 | 10 | 6 | 13 | 13 | 13 | 9 |
| Sneaks | CON | 23 | 13 | 13 | 12 | 10 | 13 | 13 | 13 | 13 | 13 | 11 | 11 | 10 | 13 | 11 | 13 | 13 | 13 | 13 |
| Sneaks | CON | 30 | 13 | 13 | 11 | 10 | 10 | 13 | 11 | 9 | 9 | 10 | 10 | 8 | 10 | 10 | 8 | 10 | 13 | 13 |
| Sneaks | CON | 37 | 13 | 8 | 8 | 10 | 10 | 10 | 13 | 10 | 10 | 8 | 10 | 10 | 8 | 8 | 10 | 13 | 13 | 10 |
| Sneaks | CON | 44 | 11 | 11 | 10 | 9 | 9 | 9 | 13 | 13 | 13 | 11 | 13 | 13 | 10 | 10 | 13 | 13 | 13 | 11 |
| Zeke | SNM | 2 | 7 | 13 | 13 | 11 | 13 | 13 | 10 | 13 | 13 | 12 | 13 | 13 | 13 | 13 | 13 | 13 | 13 | 11 |
| Zeke | SNM | 9 | 13 | 13 | 13 | 13 | 13 | 13 | 13 | 13 | 13 | 13 | 12 | 10 | 13 | 13 | 13 | 13 | 13 | 13 |
| Zeke | SNM | 16 | 13 | 13 | 13 | 13 | 13 | 13 | 13 | 13 | 13 | 13 | 13 | 13 | 13 | 13 | 13 | 13 | 13 | 13 |
| Zeke | SNM | 23 | 13 | 13 | 13 | 13 | 13 | 13 | 13 | 13 | 13 | 13 | 13 | 13 | 13 | 13 | 13 | 13 | 13 | 13 |
| Zeke | SNM | 30 | 13 | 13 | 13 | 13 | 13 | 13 | 13 | 13 | 13 | 13 | 13 | 13 | 13 | 13 | 13 | 13 | 13 | 11 |
| Zeke | SNM | 37 | 13 | 13 | 13 | 13 | 13 | 13 | 13 | 13 | 13 | 13 | 13 | 13 | 13 | 13 | 13 | 13 | 13 | 13 |
| Zeke | SNM | 44 | 13 | 13 | 13 | 13 | 13 | 13 | 13 | 13 | 13 | 13 | 13 | 13 | 13 | 13 | 13 | 13 | 13 | 13 |

* CON = Control; SNM = SORE NO-MORE ®

TABLE 2
Converted Back Scores

| | | | Converted Back Scores | | | | | | | | | | | | | | | | | | | |
|---|---|---|---|---|---|---|---|---|---|---|---|---|---|---|---|---|---|---|---|---|---|---|
| Horse | Treatment | Day | L1 | L2 | L3 | L4 | L5 | L6 | L7 | L8 | L9 | LAVG | R1 | R2 | R3 | R4 | R5 | R6 | R7 | R8 | R9 | RAVG |
| AJ | CON | 2 | 0 | 0 | 0 | 0 | 2 | 0 | 0 | 0 | 3 | 0.56 | 0 | 3 | 0 | 0 | 0 | 0 | 0 | 0 | 0 | 0.33 |
| AJ | CON | 9 | 0 | 0 | 0 | 0 | 2 | 0 | 0 | 0 | 0 | 0.22 | 0 | 0 | 0 | 0 | 0 | 0 | 0 | 0 | 0 | 0.00 |
| AJ | CON | 16 | 0 | 0 | 0 | 0 | 1 | 0 | 0 | 0 | 0 | 0.11 | 0 | 4 | 0 | 0 | 0 | 0 | 0 | 0 | 0 | 0.44 |
| AJ | CON | 23 | 0 | 3 | 3 | 0 | 0 | 0 | 0 | 0 | 0 | 0.67 | 0 | 0 | 0 | 0 | 0 | 0 | 0 | 0 | 0 | 0.00 |
| AJ | CON | 30 | 0 | 0 | 0 | 0 | 0 | 0 | 0 | 0 | 0 | 0.00 | 0 | 0 | 0 | 0 | 0 | 0 | 0 | 0 | 0 | 0.00 |
| AJ | CON | 37 | 0 | 5 | 5 | 3 | 3 | 3 | 0 | 0 | 0 | 2.11 | 3 | 3 | 3 | 3 | 3 | 3 | 3 | 3 | 3 | 3.00 |
| AJ | CON | 44 | 3 | 0 | 0 | 0 | 3 | 0 | 0 | 0 | 0 | 0.67 | 0 | 3 | 0 | 0 | 0 | 0 | 0 | 0 | 0 | 0.33 |
| Ares | SNM | 2 | 0 | 0 | 1 | 0 | 2 | 0 | 3 | 5 | 0 | 1.22 | 0 | 0 | 0 | 4 | 0 | 0 | 1 | 1 | 3 | 1.00 |
| Ares | SNM | 9 | 0 | 0 | 0 | 0 | 0 | 3 | 0 | 1 | 0 | 0.44 | 0 | 0 | 3 | 2 | 0 | 2 | 0 | 0 | 0 | 0.78 |
| Ares | SNM | 16 | 0 | 0 | 7 | 0 | 1 | 5 | 0 | 3 | 5 | 2.33 | 0 | 0 | 3 | 0 | 0 | 7 | 0 | 0 | 0 | 1.11 |
| Ares | SNM | 23 | 0 | 0 | 0 | 0 | 0 | 0 | 0 | 0 | 2 | 0.22 | 0 | 0 | 0 | 0 | 0 | 2 | 0 | 0 | 0 | 0.22 |
| Ares | SNM | 30 | 0 | 0 | 0 | 0 | 0 | 0 | 0 | 0 | 0 | 0.00 | 0 | 0 | 0 | 0 | 0 | 0 | 0 | 0 | 0 | 0.00 |
| Ares | SNM | 37 | 0 | 0 | 0 | 0 | 0 | 2 | 0 | 0 | 0 | 0.22 | 0 | 0 | 0 | 0 | 0 | 6 | 0 | 0 | 0 | 0.67 |
| Ares | SNM | 44 | 0 | 0 | 0 | 0 | 0 | 5 | 0 | 0 | 2 | 0.78 | 0 | 0 | 0 | 0 | 0 | 0 | 0 | 0 | 0 | 0.00 |
| Atticus | SNM | 2 | 7 | 8 | 0 | 5 | 9 | 9 | 7 | 8 | 9 | 6.89 | 3 | 6 | 0 | 6 | 9 | 7 | 7 | 7 | 7 | 5.78 |
| Atticus | SNM | 9 | 0 | 1 | 3 | 3 | 6 | 4 | 0 | 5 | 0 | 2.44 | 0 | 5 | 0 | 5 | 3 | 5 | 0 | 0 | 0 | 2.00 |
| Atticus | SNM | 16 | 1 | 1 | 0 | 1 | 1 | 1 | 2 | 0 | 3 | 1.11 | 1 | 1 | 0 | 1 | 0 | 1 | 1 | 0 | 0 | 0.56 |
| Atticus | SNM | 23 | 0 | 0 | 0 | 3 | 2 | 2 | 3 | 4 | 2 | 1.78 | 1 | 1 | 0 | 1 | 0 | 1 | 0 | 0 | 0 | 0.44 |
| Atticus | SNM | 30 | 0 | 2 | 0 | 0 | 0 | 2 | 0 | 2 | 0 | 0.67 | 0 | 1 | 0 | 0 | 0 | 1 | 0 | 0 | 0 | 0.22 |
| Atticus | SNM | 37 | 4 | 0 | 0 | 3 | 0 | 0 | 0 | 0 | 0 | 0.78 | 4 | 3 | 3 | 3 | 0 | 0 | 0 | 3 | 0 | 1.78 |
| Atticus | SNM | 44 | 0 | 3 | 3 | 4 | 4 | 4 | 0 | 3 | 3 | 2.67 | 5 | 5 | 5 | 5 | 7 | 3 | 0 | 3 | 0 | 3.67 |
| Champ | CON | 2 | 9 | 7 | 4 | 10 | 10 | 8 | 9 | 8 | 8 | 8.11 | 5 | 8 | 5 | 9 | 8 | 5 | 8 | 9 | 3 | 6.67 |
| Champ | CON | 9 | 6 | 6 | 3 | 5 | 6 | 5 | 2 | 4 | 4 | 4.56 | 5 | 0 | 5 | 5 | 7 | 1 | 2 | 0 | 0 | 2.78 |
| Champ | CON | 16 | 4 | 4 | 1 | 3 | 0 | 0 | 0 | 0 | 0 | 1.33 | 6 | 6 | 2 | 4 | 1 | 1 | 0 | 0 | 1 | 2.33 |
| Champ | CON | 23 | 3 | 3 | 3 | 6 | 0 | 6 | 0 | 1 | 4 | 2.89 | 5 | 5 | 3 | 7 | 0 | 0 | 1 | 3 | 0 | 2.67 |
| Champ | CON | 30 | 4 | 5 | 5 | 5 | 7 | 7 | 5 | 0 | 3 | 4.56 | 5 | 5 | 5 | 5 | 7 | 7 | 5 | 0 | 0 | 4.33 |
| Champ | CON | 37 | 5 | 5 | 5 | 3 | 3 | 3 | 3 | 0 | 3 | 3.33 | 3 | 3 | 3 | 3 | 3 | 3 | 3 | 3 | 0 | 2.67 |
| Champ | CON | 44 | 3 | 2 | 0 | 3 | 3 | 3 | 4 | 2 | 2 | 2.44 | 3 | 3 | 0 | 5 | 0 | 3 | 0 | 3 | 0 | 1.89 |
| Dublin | SNM | 2 | 4 | 8 | 6 | 8 | 9 | 8 | 9 | 8 | 6 | 7.33 | 0 | 5 | 6 | 6 | 6 | 8 | 8 | 7 | 7 | 5.89 |
| Dublin | SNM | 9 | 5 | 4 | 6 | 3 | 3 | 1 | 6 | 4 | 6 | 4.22 | 3 | 4 | 0 | 6 | 3 | 0 | 5 | 6 | 5 | 3.56 |
| Dublin | SNM | 16 | 0 | 4 | 0 | 0 | 6 | 0 | 0 | 5 | 5 | 2.22 | 4 | 5 | 0 | 6 | 5 | 7 | 6 | 6 | 0 | 4.33 |
| Dublin | SNM | 23 | 1 | 1 | 1 | 1 | 3 | 1 | 2 | 0 | 0 | 1.11 | 0 | 3 | 0 | 3 | 3 | 3 | 0 | 3 | 0 | 1.67 |
| Dublin | SNM | 30 | 0 | 0 | 0 | 5 | 0 | 0 | 3 | 3 | 3 | 1.56 | 5 | 5 | 5 | 5 | 5 | 5 | 5 | 5 | 7 | 5.22 |
| Dublin | SNM | 37 | 10 | 0 | 8 | 4 | 0 | 0 | 3 | 6 | 6 | 4.11 | 10 | 9 | 6 | 6 | 8 | 8 | 8 | 6 | 8 | 7.67 |
| Dublin | SNM | 44 | 2 | 3 | 2 | 2 | 2 | 3 | 0 | 0 | 0 | 1.56 | 2 | 3 | 2 | 3 | 2 | 2 | 0 | 0 | 2 | 1.78 |
| Fatima | CON | 2 | 2 | 6 | 5 | 4 | 0 | 2 | 0 | 6 | 0 | 2.78 | 0 | 0 | 0 | 0 | 2 | 0 | 0 | 3 | 0 | 0.56 |
| Fatima | CON | 9 | 1 | 0 | 0 | 3 | 0 | 0 | 0 | 0 | 1 | 0.56 | 4 | 0 | 4 | 0 | 0 | 0 | 0 | 0 | 1 | 1.00 |
| Fatima | CON | 16 | 4 | 1 | 6 | 1 | 0 | 0 | 0 | 1 | 0 | 1.44 | 1 | 4 | 2 | 0 | 0 | 0 | 0 | 0 | 0 | 0.78 |
| Fatima | CON | 23 | 1 | 0 | 2 | 1 | 0 | 0 | 0 | 0 | 2 | 0.67 | 0 | 0 | 3 | 0 | 2 | 1 | 0 | 1 | 0 | 0.78 |
| Fatima | CON | 30 | 1 | 4 | 3 | 2 | 1 | 0 | 3 | 0 | 3 | 1.89 | 3 | 3 | 5 | 3 | 3 | 0 | 0 | 3 | 4 | 2.67 |
| Fatima | CON | 37 | 5 | 3 | 3 | 0 | 3 | 3 | 0 | 0 | 0 | 1.89 | 6 | 7 | 6 | 0 | 2 | 0 | 0 | 5 | 0 | 2.89 |
| Fatima | CON | 44 | 3 | 0 | 3 | 0 | 3 | 0 | 0 | 0 | 0 | 1.00 | 3 | 0 | 0 | 0 | 3 | 0 | 0 | 0 | 0 | 0.67 |
| Flick | CON | 2 | 4 | 0 | 2 | 2 | 2 | 0 | 3 | 5 | 5 | 2.56 | 0 | 3 | 0 | 3 | 3 | 0 | 0 | 0 | 0 | 1.00 |
| Flick | CON | 9 | 2 | 0 | 2 | 4 | 6 | 5 | 3 | 6 | 6 | 3.78 | 0 | 3 | 0 | 0 | 2 | 0 | 3 | 4 | 7 | 2.11 |
| Flick | CON | 16 | 0 | 0 | 0 | 1 | 0 | 3 | 0 | 0 | 3 | 0.78 | 1 | 0 | 0 | 3 | 3 | 0 | 0 | 0 | 0 | 0.78 |
| Flick | CON | 23 | 0 | 0 | 0 | 2 | 2 | 1 | 0 | 2 | 1 | 0.89 | 2 | 0 | 0 | 4 | 1 | 2 | 0 | 5 | 1 | 1.67 |
| Flick | CON | 30 | 0 | 0 | 0 | 0 | 3 | 0 | 0 | 0 | 0 | 0.33 | 0 | 3 | 3 | 0 | 3 | 0 | 0 | 0 | 3 | 1.33 |
| Flick | CON | 37 | 3 | 0 | 0 | 3 | 3 | 3 | 3 | 4 | 6 | 2.78 | 4 | 5 | 5 | 6 | 3 | 2 | 3 | 3 | 3 | 3.78 |
| Flick | CON | 44 | 0 | 3 | 3 | 0 | 3 | 0 | 0 | 5 | 2 | 1.78 | 0 | 0 | 3 | 3 | 3 | 0 | 0 | 3 | 3 | 1.67 |
| Fury | CON | 2 | 3 | 0 | 2 | 3 | 7 | 5 | 0 | 0 | 0 | 2.22 | 0 | 3 | 0 | 2 | 5 | 0 | 0 | 2 | 0 | 1.33 |
| Fury | CON | 9 | 0 | 0 | 2 | 6 | 8 | 4 | 0 | 0 | 0 | 2.22 | 3 | 4 | 3 | 2 | 1 | 4 | 0 | 2 | 6 | 2.78 |
| Fury | CON | 16 | 0 | 1 | 0 | 1 | 1 | 0 | 0 | 0 | 0 | 0.33 | 0 | 1 | 0 | 0 | 0 | 3 | 0 | 1 | 3 | 0.89 |
| Fury | CON | 23 | 0 | 0 | 0 | 0 | 0 | 0 | 0 | 0 | 0 | 0.00 | 0 | 0 | 2 | 2 | 0 | 1 | 0 | 1 | 0 | 0.67 |
| Fury | CON | 30 | 0 | 0 | 0 | 0 | 5 | 3 | 0 | 0 | 0 | 0.89 | 0 | 0 | 0 | 0 | 0 | 0 | 0 | 0 | 0 | 0.00 |
| Fury | CON | 37 | 4 | 0 | 2 | 2 | 0 | 0 | 0 | 0 | 0 | 0.89 | 0 | 0 | 0 | 0 | 0 | 0 | 0 | 0 | 0 | 0.00 |
| Fury | CON | 44 | 0 | 0 | 0 | 3 | 3 | 3 | 0 | 0 | 0 | 1.00 | 0 | 0 | 0 | 3 | 0 | 3 | 0 | 0 | 0 | 0.67 |
| Glory | SNM | 2 | 5 | 10 | 6 | 8 | 10 | 8 | 10 | 10 | 7 | 8.22 | 7 | 7 | 0 | 9 | 10 | 8 | 10 | 12 | 7 | 7.78 |
| Glory | SNM | 9 | 2 | 7 | 5 | 6 | 8 | 8 | 0 | 5 | 5 | 5.11 | 4 | 6 | 2 | 0 | 3 | 5 | 0 | 5 | 6 | 3.44 |
| Glory | SNM | 16 | 5 | 5 | 5 | 5 | 5 | 5 | 0 | 5 | 5 | 4.44 | 5 | 5 | 5 | 0 | 7 | 6 | 0 | 0 | 0 | 3.11 |
| Glory | SNM | 23 | 3 | 3 | 3 | 2 | 5 | 9 | 3 | 5 | 5 | 4.22 | 5 | 5 | 5 | 5 | 3 | 7 | 0 | 5 | 5 | 4.44 |
| Glory | SNM | 30 | 0 | 0 | 0 | 0 | 0 | 0 | 0 | 0 | 0 | 0.00 | 0 | 0 | 0 | 0 | 0 | 0 | 0 | 3 | 0 | 0.33 |
| Glory | SNM | 37 | 5 | 3 | 3 | 0 | 3 | 3 | 0 | 0 | 0 | 1.89 | 3 | 0 | 0 | 0 | 0 | 0 | 0 | 0 | 0 | 0.33 |
| Glory | SNM | 44 | 2 | 4 | 0 | 0 | 0 | 0 | 0 | 0 | 2 | 0.89 | 0 | 0 | 0 | 0 | 0 | 0 | 0 | 0 | 0 | 0.00 |
| Goldie | SNM | 2 | 0 | 0 | 0 | 2 | 5 | 5 | 0 | 0 | 0 | 1.33 | 0 | 0 | 0 | 2 | 2 | 0 | 4 | 1 | 0 | 1.00 |
| Goldie | SNM | 9 | 2 | 1 | 0 | 0 | 4 | 4 | 0 | 0 | 3 | 1.56 | 1 | 2 | 3 | 2 | 6 | 4 | 2 | 5 | 5 | 3.33 |
| Goldie | SNM | 16 | 0 | 1 | 0 | 2 | 2 | 0 | 0 | 0 | 0 | 0.56 | 0 | 0 | 0 | 3 | 5 | 0 | 0 | 0 | 0 | 0.89 |
| Goldie | SNM | 23 | 0 | 0 | 0 | 0 | 3 | 0 | 0 | 0 | 0 | 0.33 | 0 | 0 | 0 | 0 | 0 | 0 | 0 | 0 | 0 | 0.00 |
| Goldie | SNM | 30 | 0 | 0 | 0 | 0 | 0 | 0 | 0 | 0 | 0 | 0.00 | 0 | 3 | 0 | 0 | 0 | 0 | 0 | 0 | 0 | 0.33 |
| Goldie | SNM | 37 | 0 | 0 | 0 | 0 | 3 | 0 | 0 | 3 | 0 | 0.67 | 0 | 3 | 3 | 0 | 3 | 0 | 0 | 0 | 0 | 1.00 |
| Goldie | SNM | 44 | 0 | 0 | 0 | 3 | 3 | 0 | 0 | 0 | 0 | 0.67 | 0 | 0 | 0 | 0 | 0 | 0 | 0 | 0 | 0 | 0.00 |
| Gypsy | CON | 2 | 1 | 4 | 0 | 4 | 4 | 4 | 4 | 5 | 3 | 3.22 | 0 | 5 | 1 | 5 | 5 | 5 | 5 | 3 | 3 | 3.56 |
| Gypsy | CON | 9 | 4 | 5 | 3 | 8 | 4 | 0 | 1 | 0 | 0 | 2.78 | 0 | 5 | 3 | 0 | 2 | 0 | 0 | 0 | 0 | 1.11 |
| Gypsy | CON | 16 | 8 | 8 | 4 | 0 | 7 | 1 | 0 | 0 | 0 | 3.11 | 8 | 10 | 0 | 3 | 8 | 0 | 0 | 0 | 1 | 3.33 |
| Gypsy | CON | 23 | 3 | 1 | 3 | 0 | 0 | 1 | 0 | 0 | 1 | 1.00 | 5 | 4 | 0 | 0 | 2 | 2 | 0 | 0 | 0 | 1.44 |
| Gypsy | CON | 30 | 8 | 6 | 4 | 0 | 3 | 3 | 0 | 4 | 3 | 3.44 | 0 | 3 | 0 | 0 | 3 | 0 | 0 | 0 | 0 | 0.67 |
| Gypsy | CON | 37 | 5 | 5 | 5 | 5 | 5 | 3 | 0 | 0 | 0 | 3.11 | 5 | 3 | 3 | 3 | 0 | 0 | 0 | 0 | 3 | 1.89 |
| Gypsy | CON | 44 | 4 | 5 | 3 | 3 | 4 | 5 | 4 | 4 | 0 | 3.56 | 0 | 4 | 4 | 3 | 4 | 5 | 2 | 2 | 2 | 2.89 |
| King | SNM | 2 | 0 | 3 | 3 | 0 | 3 | 2 | 3 | 3 | 1 | 2.00 | 0 | 1 | 1 | 0 | 1 | 2 | 0 | 0 | 0 | 0.56 |
| King | SNM | 9 | 0 | 2 | 1 | 0 | 5 | 0 | 3 | 2 | 0 | 1.44 | 3 | 2 | 0 | 2 | 0 | 2 | 0 | 4 | 0 | 1.44 |
| King | SNM | 16 | 0 | 0 | 0 | 0 | 0 | 0 | 0 | 0 | 0 | 0.00 | 3 | 0 | 0 | 3 | 2 | 0 | 3 | 0 | 0 | 1.22 |
| King | SNM | 23 | 0 | 3 | 0 | 0 | 0 | 2 | 0 | 0 | 0 | 0.56 | 2 | 0 | 0 | 0 | 0 | 0 | 0 | 0 | 0 | 0.22 |
| King | SNM | 30 | 0 | 0 | 3 | 1 | 0 | 0 | 0 | 0 | 0 | 0.44 | 3 | 3 | 0 | 0 | 0 | 0 | 0 | 0 | 0 | 0.67 |
| King | SNM | 37 | 4 | 3 | 0 | 3 | 3 | 0 | 0 | 0 | 0 | 1.44 | 3 | 3 | 0 | 4 | 0 | 0 | 5 | 0 | 0 | 1.67 |
| King | SNM | 44 | 0 | 3 | 0 | 3 | 0 | 0 | 0 | 0 | 0 | 0.67 | 3 | 0 | 0 | 0 | 3 | 0 | 0 | 0 | 0 | 0.67 |
| Nova | CON | 2 | 1 | 3 | 0 | 2 | 0 | 0 | 0 | 0 | 0 | 0.67 | 2 | 0 | 0 | 0 | 0 | 0 | 0 | 0 | 3 | 0.56 |
| Nova | CON | 9 | 2 | 5 | 2 | 0 | 0 | 2 | 0 | 0 | 0 | 1.22 | 2 | 3 | 6 | 0 | 7 | 0 | 0 | 2 | 2 | 2.44 |
| Nova | CON | 16 | 0 | 0 | 0 | 2 | 5 | 5 | 0 | 0 | 0 | 1.33 | 5 | 3 | 3 | 0 | 0 | 3 | 0 | 0 | 3 | 1.89 |
| Nova | CON | 23 | 2 | 2 | 2 | 0 | 0 | 0 | 0 | 0 | 3 | 1.00 | 4 | 0 | 3 | 0 | 0 | 0 | 0 | 0 | 0 | 0.78 |
| Nova | CON | 30 | 6 | 6 | 6 | 1 | 6 | 0 | 6 | 3 | 0 | 3.78 | 3 | 5 | 3 | 5 | 3 | 3 | 0 | 0 | 1 | 2.56 |
| Nova | CON | 37 | 3 | 3 | 3 | 3 | 3 | 3 | 3 | 3 | 3 | 3.00 | 4 | 4 | 4 | 0 | 3 | 0 | 0 | 3 | 0 | 2.00 |
| Nova | CON | 44 | 4 | 5 | 0 | 4 | 6 | 3 | 3 | 4 | 0 | 3.22 | 6 | 5 | 6 | 4 | 5 | 4 | 0 | 5 | 3 | 4.22 |
| Pierre | SNM | 2 | 0 | 6 | 6 | 9 | 8 | 9 | 8 | 8 | 7 | 6.78 | 6 | 9 | 7 | 7 | 6 | 7 | 6 | 8 | 8 | 7.11 |
| Pierre | SNM | 9 | 0 | 7 | 5 | 3 | 0 | 3 | 0 | 0 | 3 | 2.33 | 4 | 5 | 4 | 0 | 7 | 6 | 0 | 5 | 6 | 4.11 |
| Pierre | SNM | 16 | 0 | 1 | 6 | 0 | 0 | 5 | 0 | 0 | 5 | 1.89 | 4 | 0 | 5 | 2 | 5 | 3 | 3 | 0 | 5 | 3.00 |
| Pierre | SNM | 23 | 0 | 3 | 2 | 3 | 2 | 1 | 0 | 0 | 3 | 1.56 | 2 | 3 | 4 | 0 | 2 | 3 | 0 | 0 | 0 | 1.56 |
| Pierre | SNM | 30 | 0 | 2 | 2 | 0 | 0 | 4 | 0 | 0 | 0 | 0.89 | 0 | 3 | 3 | 0 | 0 | 0 | 0 | 0 | 4 | 1.11 |
| Pierre | SNM | 37 | 2 | 7 | 3 | 2 | 2 | 0 | 2 | 0 | 0 | 2.00 | 4 | 5 | 5 | 0 | 6 | 3 | 0 | 0 | 3 | 2.89 |
| Pierre | SNM | 44 | 3 | 5 | 0 | 3 | 2 | 3 | 3 | 3 | 3 | 2.78 | 3 | 3 | 2 | 3 | 0 | 3 | 0 | 0 | 2 | 1.78 |
| Quick | CON | 2 | 0 | 0 | 0 | 0 | 0 | 0 | 0 | 0 | 0 | 0.00 | 0 | 0 | 0 | 0 | 0 | 0 | 1 | 1 | 0 | 0.22 |
| Quick | CON | 9 | 0 | 0 | 0 | 0 | 0 | 0 | 0 | 0 | 0 | 0.00 | 0 | 0 | 0 | 0 | 0 | 0 | 0 | 0 | 0 | 0.00 |
| Quick | CON | 16 | 0 | 0 | 0 | 0 | 0 | 0 | 0 | 0 | 0 | 0.00 | 0 | 0 | 0 | 0 | 0 | 0 | 0 | 0 | 0 | 0.00 |
| Quick | CON | 23 | 0 | 0 | 0 | 0 | 0 | 0 | 0 | 0 | 0 | 0.00 | 0 | 0 | 0 | 0 | 3 | 0 | 0 | 0 | 0 | 0.33 |
| Quick | CON | 30 | 0 | 0 | 0 | 0 | 0 | 0 | 0 | 0 | 0 | 0.00 | 0 | 0 | 0 | 0 | 3 | 0 | 0 | 0 | 0 | 0.33 |
| Quick | CON | 37 | 0 | 0 | 0 | 0 | 0 | 0 | 0 | 0 | 0 | 0.00 | 0 | 0 | 0 | 0 | 0 | 0 | 0 | 0 | 0 | 0.00 |
| Quick | CON | 44 | 0 | 2 | 0 | 0 | 0 | 0 | 0 | 0 | 0 | 0.22 | 3 | 0 | 0 | 0 | 0 | 0 | 3 | 0 | 0 | 0.67 |
| Red | SNM | 2 | 0 | 0 | 0 | 1 | 3 | 3 | 0 | 0 | 0 | 0.78 | 0 | 0 | 3 | 2 | 2 | 2 | 4 | 3 | 4 | 2.22 |
| Red | SNM | 9 | 0 | 3 | 5 | 6 | 6 | 0 | 0 | 6 | 5 | 3.44 | 5 | 0 | 0 | 6 | 0 | 5 | 4 | 3 | 1 | 2.67 |
| Red | SNM | 16 | 0 | 3 | 3 | 3 | 6 | 4 | 0 | 1 | 3 | 2.56 | 1 | 0 | 3 | 3 | 5 | 0 | 0 | 0 | 0 | 1.33 |
| Red | SNM | 23 | 0 | 0 | 0 | 0 | 0 | 0 | 0 | 0 | 0 | 0.00 | 0 | 3 | 0 | 0 | 3 | 0 | 0 | 0 | 0 | 0.67 |
| Red | SNM | 30 | 0 | 0 | 0 | 0 | 0 | 0 | 0 | 0 | 0 | 0.00 | 0 | 3 | 0 | 0 | 0 | 0 | 0 | 0 | 0 | 0.33 |
| Red | SNM | 37 | 0 | 0 | 0 | 3 | 0 | 0 | 0 | 0 | 0 | 0.33 | 5 | 0 | 0 | 0 | 3 | 0 | 0 | 0 | 0 | 0.89 |
| Red | SNM | 44 | 0 | 0 | 0 | 0 | 0 | 0 | 0 | 0 | 0 | 0.00 | 0 | 0 | 0 | 0 | 0 | 0 | 0 | 0 | 0 | 0.00 |
| Sneaks | CON | 2 | 4 | 0 | 0 | 5 | 7 | 0 | 3 | 3 | 0 | 2.44 | 1 | 4 | 3 | 8 | 8 | 8 | 5 | 6 | 8 | 5.67 |
| Sneaks | CON | 9 | 3 | 0 | 0 | 3 | 5 | 0 | 3 | 3 | 0 | 1.89 | 0 | 3 | 3 | 7 | 7 | 6 | 3 | 3 | 8 | 4.44 |
| Sneaks | CON | 16 | 0 | 0 | 0 | 0 | 3 | 3 | 0 | 0 | 0 | 0.67 | 0 | 0 | 4 | 3 | 7 | 0 | 0 | 0 | 4 | 2.00 |
| Sneaks | CON | 23 | 0 | 0 | 1 | 3 | 0 | 0 | 0 | 0 | 0 | 0.44 | 2 | 2 | 3 | 0 | 2 | 0 | 0 | 0 | 0 | 1.00 |
| Sneaks | CON | 30 | 0 | 0 | 2 | 3 | 3 | 0 | 2 | 4 | 4 | 2.00 | 3 | 3 | 5 | 3 | 3 | 5 | 3 | 0 | 0 | 2.78 |
| Sneaks | CON | 37 | 0 | 5 | 5 | 3 | 3 | 3 | 0 | 3 | 3 | 2.78 | 5 | 3 | 3 | 5 | 5 | 3 | 0 | 0 | 3 | 3.00 |
| Sneaks | CON | 44 | 2 | 2 | 3 | 4 | 4 | 4 | 0 | 0 | 0 | 2.11 | 2 | 0 | 0 | 3 | 3 | 0 | 0 | 0 | 2 | 1.11 |
| Zeke | SNM | 2 | 6 | 0 | 0 | 2 | 0 | 0 | 3 | 0 | 0 | 1.22 | 1 | 0 | 0 | 0 | 0 | 0 | 0 | 0 | 2 | 0.33 |
| Zeke | SNM | 9 | 0 | 0 | 0 | 0 | 0 | 0 | 0 | 0 | 0 | 0.00 | 0 | 1 | 3 | 0 | 0 | 0 | 0 | 0 | 0 | 0.44 |
| Zeke | SNM | 16 | 0 | 0 | 0 | 0 | 0 | 0 | 0 | 0 | 0 | 0.00 | 0 | 0 | 0 | 0 | 0 | 0 | 0 | 0 | 0 | 0.00 |
| Zeke | SNM | 23 | 0 | 0 | 0 | 0 | 0 | 0 | 0 | 0 | 0 | 0.00 | 0 | 0 | 0 | 0 | 0 | 0 | 0 | 0 | 0 | 0.00 |
| Zeke | SNM | 30 | 0 | 0 | 0 | 0 | 0 | 0 | 0 | 0 | 0 | 0.00 | 0 | 0 | 0 | 0 | 0 | 0 | 0 | 0 | 2 | 0.22 |
| Zeke | SNM | 37 | 0 | 0 | 0 | 0 | 0 | 0 | 0 | 0 | 0 | 0.00 | 0 | 0 | 0 | 0 | 0 | 0 | 0 | 0 | 0 | 0.00 |
| Zeke | SNM | 44 | 0 | 0 | 0 | 0 | 0 | 0 | 0 | 0 | 0 | 0.00 | 0 | 0 | 0 | 0 | 0 | 0 | 0 | 0 | 0 | 0.00 |

* CON = Control; SNM = SORE NO-MORE ®

What is claimed is:

1. A method for reducing back pain in a subject, the method comprising
a) identifying a subject in need of treatment for back pain, and
b) administering composition A to the subject, wherein composition A comprises witch hazel extract and leaves, arnica (Arnica montana), rosemary (Rosmarinus officinalis), lavender (Lavendula officinalis), and lobelia (Lobelia inflata).

2. The method of claim 1, wherein the lobelia is substituted with comfrey (Symphytum officnate).

3. The method of claim 1, wherein the lavender is substituted with ginger (Zingiber officinale).

4. The method of claim 1, wherein the subject has back pain that has not resolved following a rest period.

5. The method of claim 4, wherein the rest period is about 30 days.

6. The method of claim 1, wherein the back pain is refractory back pain.

7. The method of claim 1, wherein the back pain is eliminated.

8. The method of claim 1, wherein the subject is an equine.

9. The method of claim 8, wherein the back pain is in the third quadrant of an equine.

10. The method of claim 1, wherein the composition is administered daily.

11. The method of claim 1, wherein the composition is administered for at least a week.

12. The method of claim 1, wherein the composition is administered for two weeks.

13. The method of claim 1, wherein the composition is administered for greater than two weeks.

14. The method of claim 1, wherein the composition is administered during known risk factors for back pain selected from the group consisting of heavy exercising, high stress activities, traveling, training, and competition.

15. The method of claim 1, wherein the composition is administered following exercise.

16. The method of claim 1, further comprising administering standard treatments for back pain.
